(19) United States
(12) Patent Application Publication
(10) Pub. No.: US 2024/0415456 A1
(43) Pub. Date: Dec. 19, 2024

(54) MEDICAL WATCH FOR MEASURING PHYSIOLOGICAL PARAMETERS

(71) Applicant: CARDIACSENSE LTD., Caesarea (IL)

(72) Inventors: Guy OFEK, Shimshit (IL); Andres WAINSTEIN, Ramat Gan (IL); Igor KOUPERMAN, Yokneam (IL); Boris SPEKTOR, Haifa (IL); Eldad SHEMESH, Binyamina (IL)

(21) Appl. No.: 18/700,205

(22) PCT Filed: Oct. 3, 2022
(86) PCT No.: PCT/IL2022/051054

§ 371 Date: Apr. 10, 2024

(30) Foreign Application Priority Data
Oct. 12, 2021 (IL) 287216

(51) Int. Cl. A61B 5/00; A61B 5/0205; A61B 5/1455; A61B 5/256; A61B 5/28; A61B 5/332

(52) U.S. Cl. CPC A61B 5/681; A61B 5/02055; A61B 5/256; A61B 5/28; A61B 5/332; A61B 5/14552; A61B 2560/0443; A61B 2562/164; A61B 2562/166

(57) ABSTRACT

The present disclosure provides, in one of its aspects, a continuous ECG unit couplable to a medical watch that comprises a first and second ECG sensing surfaces. The continuous ECG unit is configured to couple to a first ECG sensing surface that is formed on a peripheral portion of the watch and maintaining the second ECG sensing surface, that is formed at the bottom of the medical watch, exposed. The continuous ECG unit comprises an extended ECG electrode that is attachable to a portion body of the subject and is electrically coupled to the first ECG sensing surface, thereby extending it. This allows a subject wearing the watch to perform a continuous ECG measurement, such as in a Holter device.

### TECHNOLOGICAL FIELD

The present disclosure is in the field of medical devices, in particular wearable devices for medical use.

### BACKGROUND ART

References considered to be relevant as background to the presently disclosed subject matter are listed below:
WO 2019/215723
CN211560078
U.S. Pat. No. 20,203,75493
WO2021057873

Acknowledgement of the above references herein is not to be inferred as meaning that these are in any way relevant to the patentability of the presently disclosed subject matter.

### GENERAL DESCRIPTION

The present disclosure provides, in a first aspect, a medical watch for allowing various measurements of physiological parameters of the subject wearing it. The medical watch is designed to facilitate a simultaneous measurement of at least two types of measurements that require placing a finger on a specific spot in order to perform the measurement. For allowing the simultaneous measurement, the watch is designed with a peripheral rim or a peripheral section (used herein interchangeably throughout the application with the term “peripheral member”) that accommodates two sensing surfaces of different sensors that are located at substantially opposite sides of the peripheral rim to allow easily placing of the thumb and an additional finger simultaneously on the two sensing surfaces. For example, the two sensing surfaces can be of a pulse oximetry sensor and an ECG electrode. In this example, the medical watch is capable of performing simultaneous measurement of SpO₂ data and ECG data of the subject and correlate them. In some embodiments, the medical watch can include additional sensors, such as a PPG sensor, to collect additional physiological data from the subject and correlate between all the acquired medical data at a single time point.

As can be appreciated throughout the application, there are references to elements or surfaces that are formed on the peripheral member, both representing parts of sensor that are intended for performing measurements of physiological parameters thereby.

It is to be noted that any combination of the described embodiments with respect to any aspect of this present disclosure is applicable. In other words, any aspect of the present disclosure can be defined by any combination of the described embodiments.

Thus, a first aspect of the present disclosure provides a medical watch for measuring physiological parameters of a subject. The medical watch comprising a top part having a top face that comprises a display. A central axis is defined normal to the top face. The medical watch further comprises a bottom part having a bottom face for facing the skin of a subject when the watch is worn and a peripheral member that is defined between the top face and the bottom face or used for coupling the bottom part and the top part of the medical watch. The peripheral member comprises or accommodates a pulse oximetry sensing surface and a first ECG sensing surface. Each of the sensing surfaces faces different radial directions with respect to the central axis. Namely, each of the directions that the sensing surfaces are facing is normal to the central axis and the direction of one of them is different than the other.

In some embodiments of the medical watch, the pulse oximetry sensing surfaces and the first ECG sensing surface are located at substantially two opposite portions of the peripheral member such they are facing substantially two opposite radial directions, therefore allowing to place simultaneously two fingers on the two sensors, typically the thumb and either the index or the middle finger, for performing simultaneous measurement from the two sensors.

It is to be noted that the term “substantially” in the context of opposite directions/positions of the sensing surfaces throughout the application should be understood as ±20° deviation from opposite portions of the peripheral member or ±20° deviation of radial directions that the sensing surfaces face with respect to the central axis. In some embodiments the term “substantially” should be understood as ±20° deviation from opposite portions of the inscribed circle of the shape of peripheral member or of the circumscribed circle of the shape of the peripheral member.

In some embodiments of the medical watch, the peripheral member further comprises a temperature sensing surface for measuring the temperature of the subject.

In some embodiments of the medical watch, the bottom face comprises a second ECG sensing surface for allowing ECG measurement by the first and second ECG sensing surfaces.

In some embodiments of the medical watch, the bottom face comprises a PPG sensing surface for measuring physiological parameters from a skin portion of the wrist of the subject.

In some embodiments of the medical watch, the peripheral member has a generally ring shape and the sensing surfaces are formed on the outer sider of the ring.

In some embodiments, the medical watch further comprising a connector for coupling to a continuous ECG unit. The connector is formed in the first ECG sensing element.

In some embodiments of the medical watch, the coupling is by means of a plug formed at a proximal end of flexible connecting element that comprises one or more active leads and a ground lead that is coupled, through the plug, to a ground connection in the medical watch.

In some embodiments, the medical watch further comprising a continuous ECG unit that comprises an extended ECG electrode electronically connected by a connecting element to the first ECG sensing surface. Namely, the extended ECG electrode is physically connected by connecting elements to the first ECG sensing surface. The extended ECG electrode is practically an ECG electrode that is connectable to the first ECG sensing surface and can be placed on any desired skin portion of the subject, therefore extending the options for locating the sensing portion related to the first ECG sensing surface. Thus, the ECG sensing capabilities of the watch are extended such that the extended ECG electrode can be attached constantly to a skin portion of a subject wearing the watch to perform continuous ECG measurement by the medical watch.

In some embodiments of the medical watch, the connecting element is further coupled to a ground connection formed in the medical watch.

In some embodiments of the medical watch, the peripheral member further comprises a sensing surface of a temperature sensor, the temperature sensor comprises said ground connection.

In some embodiments of the medical watch, at least part of the connecting element is a flexible PCB. In some embodiments, the flexible PCB portion of the connecting element is the portion that is tightly fitted over the peripheral member of the medical watch.

In some embodiments of the medical watch, the continuous ECG unit comprises a casing made of or comprises an electrically insulating material. The casing is configured to (i) fit over the peripheral member and (ii) accommodate at least a part of the connecting element such that at least portions of the connecting element are disposed between the peripheral member and the casing. Thus, the casing maintains a constant contact of the connecting element with the first ECG sensing surface, and optionally also with the ground connection and prevents undesired contact of the user with the connecting element that may interrupt the ECG measurement.

In some embodiments of the medical watch, the casing is designed to allow accessibility to the pulse oximetry sensing surface. In a specific embodiment, the casing comprises an opening, an aperture or is shaped in a manner allowing to perform a measurement of the pulse oximetry sensor while being fitted over the peripheral member.

Yet another aspect of the present disclosure provides a continuous ECG unit couplable to a medical watch that comprises a first and second ECG sensing surfaces. The continuous ECG unit comprising an extended ECG electrode and a connecting element connected to the extended ECG electrode for allowing electronic connection with a first ECG sensing surface that is formed on an external portion of a watch, in particular that is formed on a peripheral member of watch.

In some embodiments of the continuous ECG unit the connecting element is further couplable to a ground connection formed in the medical watch.

In some embodiments of the continuous ECG unit, at least part of the connecting element is a flexible PCB.

In some embodiments, the continuous ECG unit further comprising a casing made of an electrically insulating material and is configured to (i) tightly fit over a peripheral member of the medical watch that comprises said first ECG sensing surface, namely fit over the peripheral rim of the medical watch such that it is secured in place: (ii) accommodate at least a part of the connecting element such that at least portions of the connecting element are disposed between the peripheral member and the casing, when being fitted over the peripheral member; and (iii) maintaining a bottom face of the medical watch, that comprises the second ECG sensing surface, exposed to allow constant contact between the second ECG sensing surface and the wrist of a subject wearing the watch with the continuous ECG unit.

In some embodiments, the portions of the connecting element that are disposed between the peripheral member and the casing are constituted by a flexible PCB.

In some embodiments of the continuous ECG unit, the casing is designed to allow access for additional one or more sensing elements of the medical watch.

In some embodiments of the continuous ECG unit, the casing comprises apertures allowing accessibility to said additional one or more sensing elements. The apertures are formed in the insulated material and are generally having the shape (may be a bit smaller or a bit larger) of the sensing elements they are allowing the access to.

In some embodiments of the continuous ECG unit, said additional one or more sensing elements comprises a pulse oximetry sensing surface and/or a temperature sensor sensing surface.

In some embodiments of the continuous ECG unit, the casing is designed to allow accessibility to the sensing surface of the pulse oximetry sensor.

In some embodiments of the continuous ECG unit, the casing comprises an opening, an aperture or is shaped in a manner allowing to perform a measurement of the pulse oximetry sensor while being fitted over the peripheral member.

Yet another aspect of the present disclosure provides a system for measuring physiological parameters of a subject. The system comprising a medical watch that comprises a top part having a top face that comprises a display. A central axis is defined normal to the top face. The medical watch further comprises a bottom part having a bottom face that faces the skin of a subject when the watch is worn, and a peripheral member defined between the top face and the bottom face or coupling the top part and the bottom part of the medical watch. A first ECG sensing surface is formed at the peripheral member and a second ECG sensing surface is formed at the bottom face. The system further comprises a continuous ECG unit of any of the above embodiments or any combination thereof, wherein the continuous ECG unit is electrically coupled to the first ECG sensing surface.

In some embodiments of the system, the connecting element is further coupled to a ground connection formed in the medical watch.

In some embodiments of the system, the peripheral member further comprises a sensing surface of a temperature sensor, and the temperature sensor comprises said ground connection.

Yet another aspect of the present disclosure provides a method for converting a medical watch to a continuous ECG measurement device. The medical watch comprises a first and second ECG sensing surfaces. The medical watch comprises a top face that comprises a display, a bottom face for contacting the skin of the subject and a peripheral member disposed therebetween. The peripheral member comprises or accommodates a first ECG sensing element, and the bottom face comprises a second ECG sensing element. The method comprises fitting a continuous ECG unit according to any one of the above-described embodiments on the peripheral member.

In some embodiments of the method, said fitting is such that the continuous ECG unit is fitted only on portions of the peripheral member.

### EMBODIMENTS

The following are optional embodiments and combinations thereof in accordance with aspects of the present disclosure:
1. A medical watch for measuring physiological parameters of a subject, comprising:
   a watch body defined between a top face that comprises a display, a bottom face for contacting the skin of the subject and a peripheral member disposed therebetween;
   wherein said peripheral member comprises or accommodates a pulse oximetry sensing element and a first ECG sensing element, each disposed on a different portion of said peripheral surface.
2. The medical watch of embodiment 1, wherein the pulse oximetry sensing element and the first ECG sensing element are located at substantially two opposite portions of said peripheral member surface.
3. The medical watch of embodiment 1 or 2, wherein said peripheral member further comprises a temperature sensing element for measuring the temperature of the subject.
4. The medical watch of any one of embodiments 1-3, wherein the bottom face comprises a second ECG sensing element for allowing ECG measurement between the first and second ECG sensing surfaces.
5. The medical watch of any one of embodiments 1-4, wherein the bottom face comprises a PPG sensing surface.
6. The medical watch of any one of embodiments 1-5, wherein the peripheral member is annular.
7. The medical watch of any one of embodiments 1-6, comprising
   a connector for coupling to a continuous ECG unit,
   said connector being formed in the first ECG sensing element.
8. The medical watch of embodiment 7, wherein the coupling is by means of a plug formed at a proximal end of flexible connecting element that comprises one or more active leads and a ground lead that is coupled, through the plug, to a ground connection in the medical watch.
9. The medical watch of embodiment 8, wherein the peripheral member further comprises a sensing element of a temperature sensor, the temperature sensor comprises said ground connection.
10. The medical watch of embodiment 8 or 9, wherein at least part of the connecting element is a flexible PCB.
11. The medical watch of any one of embodiments 7-10, wherein the continuous ECG unit comprises a casing made of an electrically non-conducting material and is configured to (i) fit over the peripheral member and (ii) accommodate at least a part of the connecting element such that at least portions of it are disposed between the peripheral member and the casing.
12. The medical watch of embodiment 11, wherein the casing is designed to allow contact with the pulse oximetry sensing element.
13. A continuous ECG unit couplable to a medical watch that comprises a first and second ECG sensing surfaces, the continuous ECG unit comprising:
   an extended ECG electrode electronically connectable by a connecting element to a first ECG sensing element of an ECG electrode that is formed on a peripheral member of a watch.
14. The continuous ECG unit of embodiment 13, wherein the connecting element is further couplable to a ground connection formed in the medical watch.
15. The continuous ECG unit of embodiment 13 or 14, wherein at least part of the connecting element is a flexible PCB.
16. The continuous ECG unit of any one of embodiments 13-15, further comprising a casing made of an electrically non-conducting material and is configured to (i) fit over a peripheral member of the medical watch that comprises said first ECG sensing surface and (ii) accommodate at least a part of the connecting element such that at least portions of the connecting element are disposed between the peripheral member and the casing, when being fitted over the peripheral member.
17. The continuous ECG unit of embodiment 16, wherein the casing is designed to allow contact with the sensing element of the pulse oximetry sensor.
18. A system for measuring physiological parameters of a subject, comprising:
   a medical watch that comprises
      a watch body defined between a top face that comprises a display, a bottom face for contacting the skin of the subject and a peripheral member disposed between the bottom and top faces:
      wherein said peripheral member comprises or accommodates a first ECG sensing element, and a second ECG sensing element is formed at the bottom face; and
   a continuous ECG unit of any one of embodiments 13-17.
19. The system of embodiment 18, wherein the ECG unit is electrically coupled to the first ECG sensing element.
20. The system of embodiment 18 or 19, wherein the connecting element is further coupled to a ground connection formed in the medical watch.
21. The system of embodiment 20, wherein the peripheral member further comprises a sensing surface of a temperature sensor, the temperature sensor comprises said ground connection.
22. A method for converting a medical watch to a continuous ECG measurement device, the medical watch comprises
a first and second ECG sensing surfaces, wherein the medical watch comprises a top face that comprises a display, a bottom face for contacting the skin of the subject and a peripheral member disposed therebetween:
wherein said peripheral member comprises or accommodates a first ECG sensing element, and the bottom face comprises a second ECG sensing element;
wherein the method comprises fitting a continuous ECG unit according to any one of claims 13-17 on the peripheral member.
23. The method of claim 22, wherein said fitting is such that the continuous ECG unit is fitted only on portions of the peripheral member.
24. The method of claim 24 or 25, wherein the medical watch is according to any one of claims 1-12.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1D are schematic illustrations of different perspective views of a non-limiting example of an embodiment of the medical watch according to an aspect of the present disclosure.

FIGS. 2A-2D are schematic illustrations of different perspective views a non-limiting example of an embodiment of the continuous ECG unit according to an aspect of the present disclosure. FIGS. 2A-2B show the continuous ECG unit alone and FIGS. 2C-2D show the continuous ECG unit coupled to a medical watch.

### DETAILED DESCRIPTION

The following figures are provided to exemplify embodiments and realization of the invention of the present disclosure.

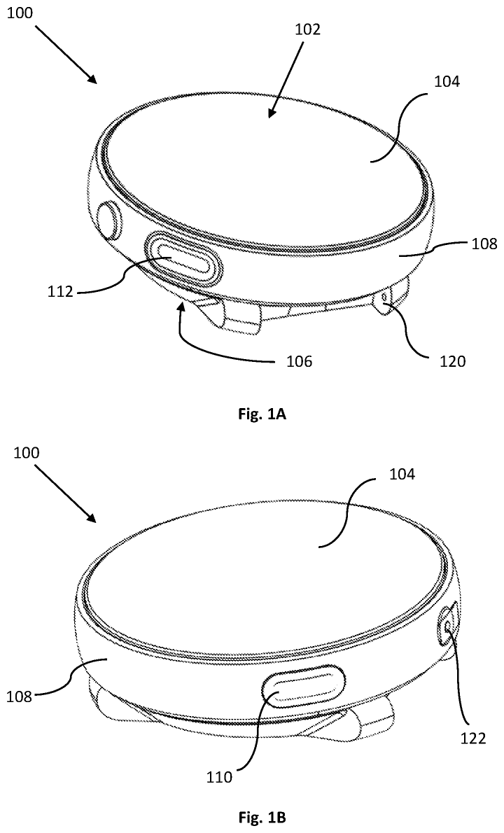

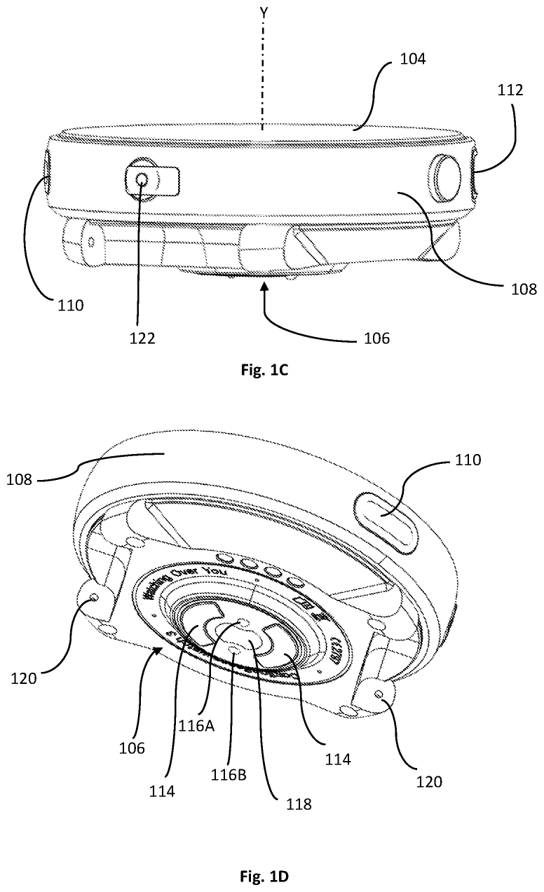

Reference is first being made to FIGS. 1A-1D, which are schematic illustrations of different perspective views of a non-limiting example of an embodiment of the medical watch according to an aspect of the present disclosure. The medical watch 100 is formed of a top face 102 that comprises a display 104 of the watch 100, a bottom face 106 and a peripheral member 108 that is defined between the top face 102 and the bottom face 106. A central axis Y is defined normal to the top face 102. The peripheral member 108 extends over the entire periphery of the medical watch 100. The peripheral member 108 comprises a first ECG sensing surface 110 of an ECG electrode and a pulse oximetry sensing surface 112 of a pulse oximetry sensor. The bottom face 106 comprises a second ECG sensing surface 114 that is designed to continuously contact the skin of the subject when the watch 100 is worn by the subject. Thus, by placing a finger on the first ECG sensing surface 110, an ECG measurement can be taken. The watch 100 is designed to allow simultaneous measurement of ECG and pulse oximetry by placing simultaneously a first finger on the first ECG sensing surface 110 and a second finger on the pulse oximetry sensing surface 112. To allow the simultaneous measurement, the first ECG sensing surface 110 and the pulse oximetry sensing surface 112 are formed at generally opposite positions of the peripheral member 108, facing substantial opposite radial directions with respect to the central axis Y. In this example, the peripheral member 108 is circular and the first ECG sensing surface 110 and the pulse oximetry sensing surface 112 are formed at generally opposite arch sections defined by the peripheral member 108.

The bottom face 106 further comprises a PPG sensor that includes two light sources 116A and 116B for illuminating the skin of the subject and a light detector 118 for receiving light from the skin in response of the illumination of the light sources. The light sources 116A and 116B can illuminate with the same wavelength range or illuminate with two different wavelength ranges. The bottom face 106 further comprises straps arrangements 120 for receiving straps to allow fastening the watch 100 over a wrist of a subject.

The peripheral member further comprises a temperature sensing surface 122 of a temperature sensor for allowing measuring of body temperature of the subject.

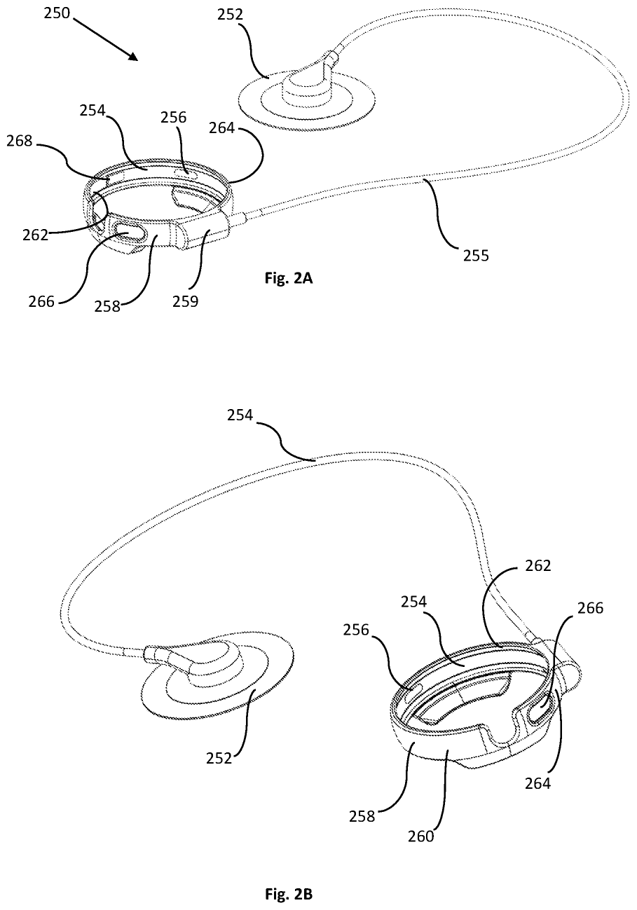

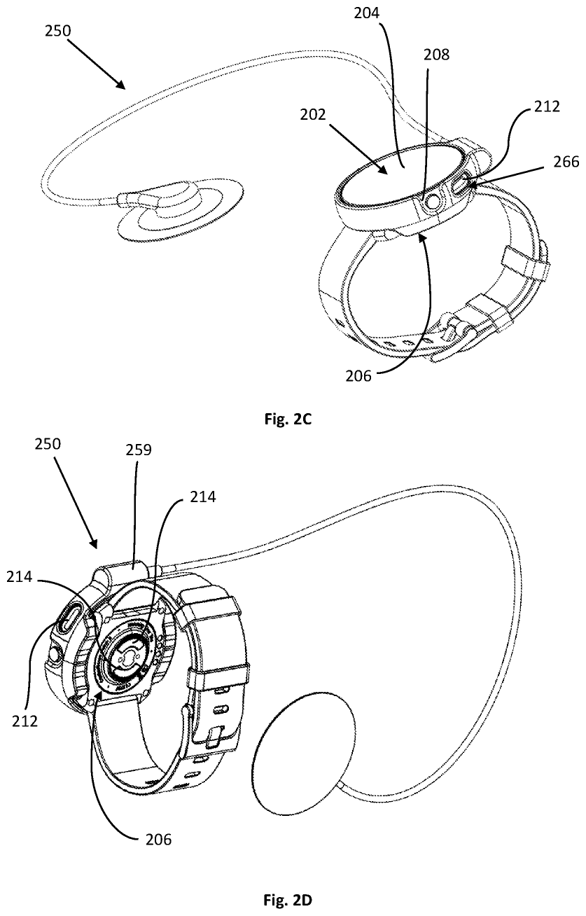

Reference is now being made to FIGS. 2A-2B, which are schematic illustrations of different perspective views a non-limiting example of an embodiment of the continuous ECG unit according to an aspect of the present disclosure. The continuous ECG unit 250 comprises an extended ECG electrode 252 that is placeable on a desired skin portion of a subject. The extended ECG electrode 252 is electronically coupled to a connecting element 254 that includes a first electronic pad 256 configured for electronically coupling with an ECG sensing surface of a watch. By electronically coupling the extended ECG electrode 252 to an ECG sensing surface of a watch, the ECG sensing surface of the watch becomes the extended ECG sensing surface. By doing that, the extended ECG electrode can be attached to a skin portion of the subject to provide a continuous ECG measurement by the watch, namely rendering the watch to a Holter device.

A part of the connecting element 254 is accommodated in an insulating casing 258 that is designed to tightly fit over the watch and is made of an electrically insulating material. Another part of the connecting element 254 is a flexible cord 255 that couples the extended ECG electrode 252 and the insulating casing 258. The insulating casing 258 comprises peripheral wall 260 having an inner side 262 to which a part of the connecting element 254 is attached to and external side 264. Thus, the insulating casing 258 insulates the connecting element 254 and prevents undesired contact between the subject and the connecting element that may interfere with the measurement. The insulating casing 258 is designed with an aperture 266 for allowing a subject to contact a sensing surface of the watch therethrough for performing a measurement, for example a pulse oximetry measurement.

The insulating casing 258 comprises a port 259 that is configured to receive the end of the flexible cord 255 to allow transition of the connecting element into the inner side 262 of the insulating casing 258.

The connecting element 254 further comprises a second electronic pad 268 for contacting a ground connection that is formed in the watch. It is to be noted that the connecting element 254 is at least partially formed of a flexible PCB.

The insulating casing 258 is designed to fit in a specific manner over a peripheral member of a watch to ensure the alignment of the electronic pads 256 and 268 with the first ECG sensing surface and the ground connection, respectively.

Reference is now being made to FIGS. 2C-2D, which show the continuous ECG unit 250 being fitted over a peripheral member of a watch. The peripheral member 208 is defined between a top face 202 that is formed with a display 204 and a bottom face 206 that includes a second ECG sensing surface 214 of a second ECG electrode. The peripheral member 208 comprises the first ECG sensing surface and the ground connection to which the electronic pads of the continuous ECG unit is connected to. The peripheral member 208 further comprises a pulse oximetry sensing surface 212 that is accessible via the aperture 266 in the insulating casing 258.

1. A continuous ECG unit couplable to a medical watch that comprises a first and second ECG sensing surfaces, the continuous ECG unit comprising:
an extended ECG electrode electronically connectable by a connecting element to a first ECG sensing element of an ECG electrode that is formed on a peripheral member of a watch;
a casing made of an insulating material and is configured to (i) tightly fit over a peripheral member of the medical watch that comprises said first ECG sensing surface, (ii) accommodate at least a part of the connecting element such that at least portions of the connecting element are disposed between the peripheral member and the casing, when being fitted over the peripheral member so as to maintain a constant contact of the connecting element with the first ECG sensing surface, and (iii) maintaining a bottom face of the medical watch that comprises the second ECG surface exposed.

2. The continuous ECG unit of claim 1, wherein the connecting element is further couplable to a ground connection formed in the medical watch.

3. The continuous ECG unit of claim 1 or 2, wherein at least part of the connecting element is a flexible PCB.

4. (canceled)

5. The continuous ECG unit of claim 1, wherein the casing is designed to allow access for additional one or more sensing elements of the medical watch.

6. The continuous ECG unit of claim 5, wherein the casing comprises apertures allowing accessibility to said additional one or more sensing elements.

7. The continuous ECG unit of claim 5 or 6, wherein said additional one or more sensing elements comprises a pulse oximetry sensing surface and/or a temperature sensor sensing surface.

8. A system for measuring physiological parameters of a subject, comprising:
a medical watch that comprises
a watch body defined between a top face that comprises a display, a bottom face for contacting the skin of the subject and a peripheral member disposed between the bottom and top faces;
wherein said peripheral member comprises or accommodates a first ECG sensing element, and a second ECG sensing element is formed at the bottom face; and
a continuous ECG unit of claim 1.

9. The system of claim 8, wherein the ECG unit is electrically coupled to the first ECG sensing element.

10. The system of claim 8 or 9, wherein the connecting element is further coupled to a ground connection formed in the medical watch.

11. The system of claim 10, wherein the peripheral member further comprises a sensing surface of a temperature sensor, the temperature sensor comprises said ground connection.

12.-23. (canceled)

24. A method for converting a medical watch to a continuous ECG measurement device, the medical watch comprises a first and second ECG sensing surfaces, wherein the medical watch comprises a top face that comprises a display, a bottom face for contacting the skin of the subject and a peripheral member disposed therebetween;
wherein said peripheral member comprises or accommodates a first ECG sensing element, and the bottom face comprises a second ECG sensing element;
wherein the method comprises fitting a continuous ECG unit according to any one of claim 1, 2, 5, or 6 on the peripheral member.

25. The method of claim 24, wherein said fitting is such that the continuous ECG unit is fitted only on portions of the peripheral member.

26. (canceled)
